(19) United States
(12) Patent Application Publication
(10) Pub. No.: US 2002/0090617 A1
(43) Pub. Date: Jul. 11, 2002

(54) Hybridisation assay in which excess probe is destroyed

(76) Inventor: Stuart Harbron, Berkhamsted (GB)

Correspondence Address: Hopgood, Calimafde, Judlowe & Mondolino, 60 East 42nd Street, New York, NY, 10165, US

(21) Appl. No.: 09/833,918

(22) Filed: Apr. 13, 2001

Related U.S. Application Data
(63) Continuation-in-part of application No. 09/403,105, filed on Oct. 14, 1999.

(30) Foreign Application Priority Data
Oct. 12, 1998 (GB) 9822067
Oct. 12, 1999 (GB) PCT/GB99/03383

(51) Int. Cl. C12Q 1/68

(52) U.S. Cl. 435/6

(57) ABSTRACT

The present invention provides a method for detecting a single-stranded target nucleic acid comprising the steps of: a) forming a hybrid between a target nucleic acid and a nucleic acid probe, said nucleic acid probe labelled with an enzyme reagent which hydrolyzes single-stranded nucleic acid but is substantially without effect on double-stranded nucleic acid, said hybrid formed under conditions of pH which are outside the activity range of said enzyme reagent; b) adjusting said pH to a value within the activity range of said enzyme reagent, whereby said enzyme reagent substantially hydrolyzes any single-stranded nucleic acid present; and c) contacting said hybrid with a detection reagent to detect the hybrid, characterized by, prior to step (c), bringing the nucleic acid probe or hybrid into contact with a solid support to attach it thereto or bringing the nucleic acid probe or hybrid into contact with a capture reagent, optionally linked to a solid support, to capture the nucleic acid probe or hybrid; and washing the capture reagent or solid support on which the hybrid is immobilized with a washing fluid while the capture reagent or solid support is contained within a vessel that is adapted to retain the capture reagent or solid support but not to retain fluid in which the capture reagent or solid support is dispersed, whereby material which has not been captured by the capture reagent or otherwise immobilized on a solid support is eluted from the vessel.

### TECHNICAL FIELD

This invention relates to methods for detecting nucleic acids.

### BACKGROUND ART

Nucleic acid hybridisation is a widely used technique for identifying, detecting and quantitating target polynucleotide sequences in a sample. This technique relies for its success on complementary base pairing between the two halves of a double-stranded nucleic acid molecule: when single-stranded nucleic acids are incubated in solution under suitable conditions of temperature, pH and ionic strength, complementary base sequences pair to form double-stranded stable hybrid molecules. This ability of single-stranded nucleic acid molecules to form a hydrogen-bonded structure with their complementary nucleic acid sequences has long been employed as an analytical tool in recombinant DNA research.

In most cases the sample will contain double-stranded nucleic acid and must be denatured prior to the hybridisation assay to render it single-stranded. A nucleic acid having a known sequence which is complementary to the target sequence is either synthesised chemically in an automated fashion with great facility, or is isolated from the appropriate organism and rendered single-stranded by denaturation. It is then used as a probe to search a sample for a target complementary sequence. Detection of specific target nucleic acids enables accurate diagnosis of bacterial, fungal and viral disease states in humans, animals and plants. Additionally, the ability to probe for a specific nucleotide sequence enables the diagnosis of human genetic disorders. Hybridisation produces stable hybrids, and a number of different approaches are known to the art for detecting these.

One approach involves the use of labelled probes. By labelling a probe nucleic acid with some readily detectable chemical group, it is possible to detect the polynucleotide sequence of interest in a test medium containing sample nucleic acids in single-stranded form. Nucleic acids have been labelled with radioisotopes, enzymes and fluorescent molecules. The use of labelled nucleic acids as probes in macromolecular analysis is important for clinical, veterinary and environmental diagnostic applications.

It is important that enzymes employed as labels catalyse a reaction which has an easily detectable product, and have a high turnover number to allow sensitive detection: horseradish peroxidase and alkaline phosphatase are most common. Although sensitive chemiluminometric assays for horseradish peroxidase have been described which allow small amounts of enzyme to be detected, problems associated with its use include lack of enzyme and substrate stability and the presence of endogenous peroxidases in samples.

For alkaline phosphatase, enzyme amplification cycles have been described which further reduce the amount of enzyme which can be detected, thereby extending the detection limit. In one method, the amplification system comprises an apoenzyme which is convertible into a holoenzyme by interaction with an accessory subunit and a masked form of the subunit which is convertible into its active unmasked form by the action of the enzyme to be detected. For example, in U.S. Pat. No. 5,445,942 to Rabin et al., a method is disclosed for detecting a hydrolase enzyme able to hydrolyse a synthetic derivative of FAD substituted in such a way that it yields FAD when hydrolysed, and is incorporated herein by reference in its entirety. Here the subunit is FAD and the masked form is 3′FADP, and the apoenzyme is apo-glucose oxidase or apo-D-aminoacid oxidase.

The FAD produced forms an active holoenzyme from the corresponding apoenzyme. This approach allows the detection of small amounts of alkaline phosphatase in short periods of time. For example, using such an amplification system in which the apoenzyme is apo-D-amino acid oxidase has permitted the detection of 0.1 amol of alkaline phosphatase in less than 30 minutes (Harbron et al., Anal. Biochem. (1992) 206: 119-124). In WO98/19168, incorporated herein by reference in its entirety, this approach is further extended to an amplification assay for nuclease P₁.

Alternatives to the use of sensitive techniques to detect small amounts of nucleic acids amplify the number of nucleic acid molecules to be detected.

In U.S. Pat. Nos. 4,683,195 and 4,683,202, DNA or RNA is amplified by the polymerase chain reaction (PCR). These patents are incorporated herein by reference in their entirety. This method involves the hybridisation of an oligonucleotide primer to the 5′ end of each complementary strand of the double-stranded target nucleic acid. The primers are extended from the 3′ end in a 5′-3′ direction by a DNA polymerase which incorporates free nucleotides into a nucleic acid sequence complementary to each strand of the target nucleic acid. After dissociation of the extension products from the target nucleic acid strands, the extension products become target sequences for the next cycle. In order to obtain satisfactory amounts of the amplified DNA, repeated cycles must be carried out, between which cycles, the complementary DNA strands must be denatured under elevated temperatures.

A method of detecting a specific nucleic acid sequence present in low copy in a mixture of nucleic acids, called ligase chain reaction (LCR), has also been described. WO89109835 describes this method and is incorporated herein by reference in its entirety. Target nucleic acid in a sample is annealed to probes containing contiguous sequences. Upon hybridisation, the probes are ligated to form detectable fused probes complementary to the original target nucleic acid. The fused probes are disassociated from the nucleic acid and serve as a template for further hybridisation's and fusions of the probes, thus amplifying geometrically the nucleic acid to be detected. The method does not use DNA polymerase.

Other known nucleic acid amplification procedures include transcription-based amplification systems (Kwoh et al., Proc. Natl. Acad. Sci. (U.S.A.) (1989) 86:1173; Gingeras et al., WO88110315; Davey et al., EP 329,822; Miller et al., WO 89/06700), RACE (Frohman, In: PCR Protocols: A Guide to Methods and Applications, Academic Press, NY (1990)) and one-sided PCR (Ohara, et al., Proc. Natl. Acad. Sci. (U.S.A.) (1989) 86:5673-5677). Particularly suitable amplification procedures include Nucleic Acid Sequence-Based Amplification, Strand Displacement Amplification, and Cycling Probe Amplification.

Methods based on ligation of two (or more) oligonucleotides in the presence of nucleic acid having the sequence of the resulting di-oligonucleotide, thereby amplifying the di-oligonucleotide, are also known (Wu et al., Genomics (1989) 4:560).

An isothermal amplification method has been described in which restriction endonucleases and ligases are used to achieve the amplification of target molecules that contain nucleotide 5′-[a-thio]triphosphates in one strand of a restriction site (Walker et al., Proc. Natl. Acad. Sci. (U.S.A.) (1992) 89:392-396).

Target amplification techniques described in the foregoing are generally complex and susceptible to inhibition, contamination and amplification of the wrong target. The somewhat simpler approach of sandwich hybridisation therefore remains attractive.

Many hybridisation techniques employ a sandwich approach using two probes: a reporter probe and a capture probe. The reporter probe is a nucleic acid having a sequence complementary to at least part of the target sequence and which is labelled with a detectable group. The capture probe is a nucleic acid having a sequence complementary to at least part of the target sequence, but which is different to that of the reporter probe, and which is labelled with an immobilisable group. In many applications, pairs of specific binding members (sbm's) have been used for this purpose.

Disadvantages of these approaches include the increased cost and complexity of using two probes. For example, for each assay two probes need to be synthesised and labelled: one for use as the capture probe, and the other for use as a reporter probe. In addition, hybridisation conditions have to be carefully chosen to form the sandwich of target, capture probe and reporter probe.

Simpler approaches that avoid the use of a capture probe have been described. Atlas and Steffan (Biotechniques (1990) 8:316-318) disclose a solution hybridisation method for detecting genetically-engineered micro-organisms in environmental samples. The detection method involves recovery of DNA from the microbial community of an environmental sample followed by hybridisation in solution with a radio-labelled RNA gene probe. After nuclease digestion of non-hybridised probe RNA, the DNA-RNA hybrids formed in the solution hybridisation are separated by column chromatography and detected by liquid scintillation counting. A similar approach is disclosed in JP04135498A in which hybrids are treated to remove any single-stranded material present prior to purification of the hybrid by gel filtration chromatography, with subsequent detection by non-radioactive techniques.

A less cumbersome approach is disclosed in U.S. Pat. No. 4,978,608 to Kung and Nagainis in which DNA is detected in a sequence non-specific manner using a high affinity single-stranded DNA-binding protein. This approach is extended in U.S. Pat. No. 5,536,648 to Kemp et al. who disclose an amplified DNA assay using a double stranded DNA binding protein. The method uses a PCR primer having a nucleotide sequence which is a ligand for a double stranded DNA-binding protein. After amplification the amplified target is captured by the double stranded DNA-binding protein immobilised on a solid surface. This method does not use a capture probe and will detect any amplification product containing the sequence which is a ligand for the double stranded DNA-binding protein. A disadvantage of this approach is that it relies on the accuracy of the amplification step for its specificity.

Another method is disclosed in U.S. Pat. No. 4,968,602 to Dattagupta. The test sample is modified chemically to introduce a reactive site. This mixture is then contacted with a reporter probe. After a solution phase hybridisation step, the hybrid is brought into contact with a surface having an immobilised reaction partner which reacts with the reactive site, and allows the unhybridised material to be washed away. A disadvantage of this approach is that the initial reaction step may interfere with the subsequent formation of the hybrid.

A further approach in which the hybrid itself is a hapten and which therefore only requires one probe is described by Carrico. In U.S. Pat. No. 4,743,535 is disclosed a nucleic acid hybridisation assay involving a reporter probe which results in the formation of a hybrid having epitopes for an antibody reagent. The antibody reagent is selective for DNA-RNA or RNA-RNA hybrids over the single-stranded nucleic acids. U.S. Pat. No. 5,200,313 to Carrico further discloses a nucleic acid hybridisation assay employing an immobilised or immobilisable polynucleotide probe selected to form DNA-RNA or RNA-DNA hybrids with the particular polynucleotide sequence to be determined. Resulting hybrids are detected by binding of an antibody reagent, preferably labelled with a detectable chemical group, selective for binding the hybrids in the presence of the single-stranded sample and probe nucleic acids. Advantageous feature of Carrico's inventions are that no immobilisation or labelling of sample nucleic acids is required and hybridisation can be performed entirely in solution. A further advantage is that a universal detection reagent may be used whatever the target is.

The key feature of Carrico's invention is the requirement for antibodies specific for double-stranded hybrids having little affinity for single-stranded nucleic acid. The generation of specific polyclonal antibodies that will bind double-stranded nucleic acid but not single-stranded nucleic acid is complicated by the fact that polyclonal antisera may contain antibodies that will cross-react with single-stranded nucleic acid. Polyclonal antisera may also contain naturally occurring antibodies to single-stranded nucleic acid or antibodies to single-stranded nucleic acid arising as a result of the immunisation. Monoclonal antibody technology can provide a means to select an antibody with desired affinity and specificity which will overcome in part these problems. Such monoclonal antibodies which will selectively bind double-stranded DNA (U.S. Pat. No. 4,623,627) or DNA-RNA hybrids (U.S. Pat. No. 4,833,084 to Carrico) have been prepared. Monoclonal antibodies are however more expensive to produce and generally have lower affinities than polyclonal antibodies.

One approach to overcome the problem of cross-reactivity is disclosed in U.S. Pat. No. 5612,458 to Hyldig-Nielson and Pluzek They use antibodies to complexes between peptide nucleic acid (PNA) and nucleic acids, particularly antibodies to nucleic acid probe-DNA or nucleic acid probe-RNA hybrids.

Another approach is to attempt to improve the affinity or selectivity of the antibody used. Fliss et al. (Applied and Environmental Microbiology (1993) 59:2698-2705) disclose murine monoclonal antibodies specific for DNA-RNA hybrids which are used to detect Lysteria DNA-RNA hybrids formed in solution from a biotinylated gene probe and rRNA extracted from Lysteria.

A further approach, disclosed in EP04055592A, attempts to combine the approach of Carrico with that of Atlas and Stefan: hybrids are treated with a reagent which removes single-stranded nucleic acids, typically a nuclease, and then detected using antibodies specific for the hybrids. A disadvantage of this approach is the possibility of contaminating the hybridisation mixture with the nuclease enzyme, which will result in the digestion of both the single stranded target and the probe before hybrids have had a chance to form, thereby reducing drastically the overall sensitivity of the assay. In addition Fliss et al. teach that the endonuclease digestion approach used by Atlas and Stefan (see above) does not efficiently separate hybridised from unhybridised molecules. Significantly, Fliss et al. do not teach that treatment with a nuclease to remove any single-stranded nucleic acids prior to capture with the murine monoclonal antibodies specific for DNA-RNA hybrids would offer an improvement to their assay.

A still further approach to eliminating problems associated with potential cross-reactivity of antibodies used in these assays is to use a probe labelled with one member of a specific binding pair, which can be captured with the second member of the pair after hybridisation and treatment with a nuclease. This approach is disclosed in EP0780479A. But again, this suffers from the inability of the endonuclease digestion approach (see above) to efficiently separate hybridised from unhybridised molecules, and problems associated with contamination of the hybridisation mixture with a nuclease, also noted above.

However, the successful use of highly sensitive enzyme amplification systems requires that background arising from non-specific association of the enzyme-labelled probe with the target nucleic acid, other reaction components, or the surface of the reaction vessel used in the assay must be substantially eliminated.

A hybridisation assay, in which excess probe is destroyed, is disclosed in GB2324307A, incorporated herein in its entirety by reference. According to this method, a nucleic acid probe is used which comprises a sequence complementary to a target nucleic acid and an enzyme reagent able to hydrolyse single-stranded nucleic acid, but which is substantially without effect on double-stranded nucleic acid. As Hybridisation between this nucleic acid probe and the target nucleic acid is performed at a pH outside the activity range of the enzyme reagent, This arrangement eliminates the possibility of contaminating the hybridisation mixture with active nuclease. the enzyme reagent is no active at the pH utilised in the hybridisation step. After hybridisation, the pH is adjusted to a value within the activity range of the enzyme reagent, and single stranded nucleic acid, including unhybridised probe, is hydrolysed. The attachment of the enzyme reagent directly to the probe facilitates hydrolysis of unhybridised probe and sterically hinders hydrolysis of hybrids, which overcomes the inability of the endonuclease digestion approach used by Atlas and Stefan and others to efficiently separate hybridised from unhybridised molecules, as observed by Fliss et al. The hybrid may be captured by an agent, which may be an antibody or nucleic acid binding protein specific for the double-stranded hybrid, or it may be one member of a pair of specific binding members, the other member attached to the hybrid. The agent itself may be immobilised or immobilisable. Alternatively, the nucleic acid probe may itself be immobilised or immobilisable.

It has now been found that the general method disclosed in the above invention may be further improved by adapting it for use with reagents immobilised onto a suitable material contained in a column. A column-based procedure not only allows more efficient washing of the bound hybrid to remove unbound components, but is also advantageously amenable to automation.

### DISCLOSURE OF INVENTION

Broadly, the present invention discloses a new and improved method for detecting single-stranded target nucleic acid.

In accordance with a first aspect of the present invention there is provided a method for detecting a single-stranded target nucleic acid comprising the steps of:

(a) forming a hybrid between a target nucleic acid and a nucleic acid probe, said nucleic acid probe labelled with an enzyme reagent which hydrolyses single-stranded nucleic acid but is substantially without effect on double-stranded nucleic acid, said hybrid formed under conditions of pH which are outside the activity range of said enzyme reagent,

(b) adjusting said pH to a value within the activity range of said enzyme reagent, whereby said enzyme reagent substantially hydrolyses any single-stranded nucleic acid present; and

(c) contacting said hybrid with a detection reagent to detect the hybrid, characterised by, prior to step (c), bringing the nucleic acid probe or hybrid into contact with a solid support to attach it thereto or bringing the nucleic acid probe or hybrid into contact with a capture reagent, optionally linked to a solid support, to capture the nucleic acid probe or hybrid; and washing the capture reagent or solid support on which the hybrid is immobilised with a washing fluid while the capture reagent or solid support is contained within a vessel that is adapted to retain the capture reagent or solid support but not to retain fluid in which the capture reagent or solid support is dispersed, whereby material which has not been captured by the capture reagent or otherwise immobilised on a solid support is eluted from the vessel.

In a first preferred embodiment, the method comprises the steps of:

(a) forming a hybrid between a target nucleic acid and a nucleic acid probe, said nucleic acid probe labelled with an enzyme reagent which hydrolyses single-stranded nucleic acid but is substantially without effect on double-stranded nucleic acid, said hybrid formed under conditions of pH which are outside the activity range of said enzyme reagent,

(b) adjusting said pH to a value within the activity range of said enzyme reagent, whereby said enzyme reagent substantially hydrolyses any single-stranded nucleic acid present,

(c) contacting said hybrid with a capture reagent, optionally linked to a solid support, whereby said hybrid is captured,

(d) introducing said capture reagent into a vessel able to retain the capture reagent but not able to retain a solution in which said capture reagent is dispersed,

(e) washing said capture reagent contained in said vessel with a washing solution, whereby material which has not been captured by said capture reagent is eluted from said vessel,

(f) contacting said hybrid with a detection reagent.

In a second preferred embodiment the method comprises the steps of:

(a) forming a hybrid between a target nucleic acid and a nucleic acid probe, said nucleic acid probe labelled with an enzyme reagent which hydrolyses single-stranded nucleic acid but is substantially without effect on double-stranded nucleic acid, said hybrid formed under conditions of pH which are outside the activity range of said enzyme reagent,

(b) adjusting said pH to a value within the activity range of said enzyme reagent, whereby said enzyme reagent substantially hydrolyses any single-stranded nucleic acid present,

(c) contacting said hybrid with a capture reagent, optionally linked to a solid support, contained in a vessel able to retain the capture reagent but not able to retain a solution in which said capture reagent is dispersed, whereby said hybrid is captured,

(d) washing said capture reagent contained in said vessel with a washing solution, whereby material which has not been captured by said capture reagent is eluted from said vessel,

(e) contacting said hybrid with detection reagent.

In a third preferred embodiment the method comprises the steps of:

a) forming a hybrid between said target nucleic acid and a nucleic acid probe, said nucleic acid probe labelled with an enzyme reagent which hydrolyses single-stranded nucleic acid but is substantially without effect on double-stranded nucleic acid, said hybrid formed under conditions of pH which are outside the activity range of said enzyme reagent, said nucleic acid probe attached to a solid support,

b) adjusting said pH to a value within the activity range of said enzyme reagent, whereby said enzyme reagent substantially hydrolyses any single-stranded nucleic acid present,

c) introducing said support into a vessel able to retain the support but not able to retain a solution in which said support is dispersed,

d) washing said support contained in said vessel with a washing solution, whereby material not attached to said support is eluted from said vessel,

e) contacting said hybrid with a detection reagent.

In a fourth preferred embodiment the method comprises the steps of:

a) forming a hybrid between said target nucleic acid and a nucleic acid probe, said nucleic acid probe labelled with an enzyme reagent which hydrolyses single-stranded nucleic acid but is substantially without effect on double-stranded nucleic acid, said hybrid formed under conditions of pH which are outside the activity range of said enzyme reagent, said nucleic acid probe attached to a solid support contained in a vessel able to retain the support but not able to retain a solution in which said support is dispersed,

b) adjusting said pH to a value within the activity range of said enzyme reagent, whereby said enzyme reagent substantially hydrolyses any single-stranded nucleic acid present,

c) washing said support contained in said vessel with a washing solution, whereby material which has not been captured by said capture means is eluted from said column,

d) contacting said support means with a detection reagent.

In a further aspect, the invention provides a variety of detection means for detecting the hybrid. The detection means may be a hybrid-binding reagent such as an antibody or DNA-binding protein specific for double-stranded nucleic acid. The detection means may also be a pair or pairs of specific binding members. These may be an antigen or hapten and the corresponding antibody; biotin and avidin, streptavidin or neutravidin, or a nucleic acid binding protein specific for a sequence present in the nucleic acid probe. Any of these agents may be labelled with a detectable label, which may an enzyme, a fluorescent moiety, a chemiluminescent moiety, an electro-chemiluminescent moiety or a coloured moiety.

In another further aspect the invention provides a variety of capture means. The capture means may be a hybrid-binding reagent such as an antibody or DNA-binding protein specific for double-stranded nucleic acid. The capture means may also comprise one member of a pair or pairs of specific binding members. These may be an antigen or hapten and the corresponding antibody; biotin and avidin, streptavidin or neutravidin; or a nucleic acid binding protein specific for a sequence present in the nucleic acid probe. These agents are attached to an insoluble support either directly or indirectly by means of an immobilisable label. The capture means may also be a material which binds nucleic acids relatively non-specifically, including silica materials, ion-exchange materials, hydrophobic materials, materials used in reversed-phase chromatography, and the like.

In another further aspect the invention provides a variety of support means. These include agaroses and their derivatives, polyacrylamides and their derivatives, magnetic materials, cellulosic materials, acrylic materials and the like.

In a further aspect the invention discloses a method for detecting DNA-RNA hybrids, DNA-DNA, RNA-RNA, DNA-RNA or DNA-PNA hybrids between a target nucleic acid and a nucleic acid probe having a sequence complementary to part of the target nucleic acid.

In a another further aspect the invention discloses a method for detecting hybrids between nucleic acid amplification products and a nucleic acid probe having a sequence complementary to part of the amplified nucleic acid.

In a further aspect the invention discloses a method for detecting hybrids between target nucleic acid extracted from a clinical specimen, a veterinary specimen, a food specimen or an environmental sample and a nucleic acid probe having a sequence complementary to part of the target nucleic acid.

In further aspects the invention provides a kit for carrying out the method.

Preferred embodiments of the invention may enable one to achieve one or more of the following objects and advantages:

(a) to provide a column-based method for detecting hybrids between a target nucleic acid and a nucleic acid probe having a sequence complementary to part of the target nucleic acid, in which any single-stranded nucleic acid is removed by treatment with an enzyme reagent attached to said probe and which is specific for single-stranded nucleic acids. Advantages of the present invention are: only a single probe is required; efficient washing is achieved using a column format; the method may be easily and advantageously automated; highly sensitive detection systems, such as chemiluminescence or enzyme amplification cascades may be used to detect the hybrids; and the sensitive detection of target nucleic acid may be achieved without using target amplification techniques, such as PCR or LCR.

(b) to provide a universal method for detecting target nucleic acid. An advantage of one embodiment is that a single type of capture agent attached to a column material may be used for any analyte; an advantage of other embodiments is that a single detection reagent may be used.

(d) to provide a method for detecting hybrids between DNA-RNA, RNA-DNA, RNA-RNA, RNA-PNA and DNA-PNA hybrids by appropriate selection of the hybrid binding reagent and enzyme reagent used.

The vessel in which the hybrid is retained in the method is preferably a column and suitably of the type used for column chromatography or it may, for example, comprise a syringe or other open ended vessel with a filter or other means for retaining the hybrid that is immobilised on the capture reagent or solid support during the washing step.

### BRIEF DESCRIPTION OF DRAWINGS

Fig. 1 is a diagrammatic representation of three preferred embodiments of the present invention for the detection of single-stranded nucleic acids.

FIG. 2 shows a standard curve for the 3′FADP-based enzyme amplification assay of nuclease P₁ (filled triangles) and nuclease S₁ (filled squares). The abscissa represents the amount of each enzyme present in amol (10⁻¹⁸ mol), and the ordinate represents the absorbance obtained after 15 mins incubation at 25° C. after subtraction of the blank reading. Both scales are logarithmic. The dotted line represents the detection limit.

### REFERENCE NUMERALS USED IN THE DRAWINGS

2—single-stranded target nucleic acid

4—nucleic acid probe

6—enzyme reagent

8—first member of a specific binding pair

10—support material

12—hybrid-binding agent

14—second member of a specific binding pair

16—product

### BEST MODES FOR CARRYING OUT THE INVENTION

The present invention provides a column-based method for detecting hybrids formed between a target nucleic acid and a nucleic acid probe. The probe is labelled with (ie has joined to its nucleic acid sequence) an enzyme reagent specific for single-stranded nucleic acids, which hydrolyses all unhybridised single-stranded nucleic acids present: this means that the hybridisation assay may be performed using only one probe. In one embodiment, the probe is attached to a column material; in another, a capture reagent is attached to a column material. In both cases, the use of a column format increases the efficiency of the washing steps needed to remove hydrolysed materials from the column.

The target nucleic acid may be DNA or RNA, and is obtained from any medium of interest, for example, a liquid sample of medical, veterinary, environmental, or industrial significance. The target nucleic acid may also be the product of a nucleic acid amplification assay, such as PCR or LCR. If the target nucleic acid is principally double stranded, it will be treated to denature it prior to the formation of the hybrid. Denaturation of nucleic acids is preferably accomplished by heating in boiling water or alkali treatment (e.g., 0.1 N sodium hydroxide), which if desired, can simultaneously be used to lyse cells. Also, release of nucleic acids from cellular or viral sources can, for example, be obtained by mechanical disruption (freeze/thaw, abrasion, sonication), physical/chemical disruption (detergents such as Triton™, Tween, or sodium dodecylsulfate, alkali treatment, osmotic shock, or heat), or enzymatic lysis (lysozyme, proteinase K, pepsin). The resulting test medium will contain the target nucleic acid in single-stranded form.

The nucleic acid probe may be a DNA probe an RNA probe, or a PNA probe. The nucleic acid probe will comprise at least one single-stranded base sequence substantially complementary to at least part of the target nucleic acid sequence. However, such base sequence need not be a single continuous polynucleotide segment, but can be comprised of two or more individual segments interrupted by non-complementary sequences. These non-hybridisable sequences are linear. In addition, the complementary region of the nucleic acid probe can be flanked at the 3′-and 5′-termini by non-hybridisable sequences, such as those comprising the DNA or RNA of a vector into which the complementary sequence had been inserted for propagation. In either instance, the nucleic acid probe as presented as an analytical reagent will exhibit detectable hybridisation at one or more points with target nucleic acids of interest. The nucleic acid probe sequence can be of any convenient or desired length, ranging from as few as a dozen to as many as 10,000 bases, and including oligonucleotides having less than about 50 bases. The nucleic acid probe may be an oligonucleotide produced by solid-phase chemistry by a nucleic acid synthesiser. The RNA or DNA probe can be obtained in a variety of other conventional manners. It should be understood that in using the expressions RNA probe and DNA probe herein, it is not implied that all nucleotides comprised in the probe be ribonucleotides or 2′-deoxyribonucleotides. Therefore, one or more of the 2′-positions on the nucleotides comprised in the probe can be chemically modified provided the antibody binding characteristics necessary for performance of the present assay are maintained to a substantial degree. Likewise, in addition or alternatively to such limited 2′-deoxy modification, the nucleic acid probe can have in general any other modification along its ribose phosphate backbone provided there is no substantial interference with the specificity of the antibody to the double stranded hybridisation product compared to its individual single strands. In preferred embodiments, in addition to the enzyme label, the nucleic acid probe is labelled with either a detectable moiety or an immobilisable moiety. For example, the nucleic acid probe is prepared by solid-phase chemistry using a nucleic acid synthesiser and has a trityl-hexyl thiol derivatised 5′-end. The covalent attachment of the label to this moiety may be achieved by a number of well-known methods using a wide range of heterobifunctional reagents. For example, the method of Carlsson et al. (Biochem J (1978) 173: 723-737) may be used: the label is reacted with 3-[(2)-pyridyldithio]propionic acid N-hydroxysuccinimide ester (SPDP) to give a 2-pyridyl disulphide-activated label. This allows disulphide exchange with trityl-hexyl thiol derivatised described above to yield a labelled nucleic acid probe. Other approaches for labelling the nucleic acid probe will be apparent to one skilled in the art. Additionally, a wide range of labelled nucleic acids is available from commercial sources. Preferred labels include the enzymes alkaline phosphatase, peroxidase, galactosidase, nuclease P₁, nuclease S₁ and mung bean nuclease; the haptens digoxin, digoxygenin, fluorescein, fluorescein isothiocyanate; and biotin or biotin analogues.

A preferred embodiment of the present invention employs a nuclease as the enzyme reagent. A number of nucleases are known which are specific for single-stranded nucleic acids. For example, ribonuclease A and ribonuclease T₁ may be used in combination to hydrolyse single-stranded RNA. Other preferred nucleases include exodeoxyribonuclease I (E.C. 3.1.11.1, similar enzymes: mammalian DNase III, exonuclease IV, T2-and T4-induced exodeoxyribonucleases), exodeoxyribonuclease (phage sp3-induced) (E.C. 3.1.11.4, exodeoxyribonuclease V (E.C. 3.1.11.5, similar enzyme: Haemophilus influenzae ATP-dependent DNase), exodeoxyribonuclease VII (E.C. 3.1.11.6, similar enzyme: Micrococcus luteus exonuclease), exoribonuclease II (E.C. 3.1.13.1, similar enzymes: RNase Q, RNase BN, RNase PIII, RNase Y), venom exonuclease (E.C. 3.1.15.1, similar enzymes: hog kidney phosphodiesterase, Lactobacillus exonuclease), spleen exonuclease (E.C. 3.1.16.1, similar enzymes: Lactobacillus acidophilus nuclease, B subtilis nuclease, salmon testis nuclease), deoxyribonuclease IV (phage T4-induced) (E.C. 3.1.21.2, similar enzymes: DNase V (mammalian, Aspergillus sojae DNase, B subtilis endonuclease, T4 endonuclease III, T7 endonuclease I, Aspergillus DNase K2, Vaccinia virus DNase VI, yeast DNase, Chlorella DNase), Aspergillus deoxyribonuclease K1 (E.C. 3.1.22.2, Aspergillus nuclease S1 (E.C. 3.1.30.1, similar enzymes: N crassa nuclease, mung bean nuclease, Penicillium citrinum nuclease P₁). Particularly preferred nucleases are nuclease P₁, nuclease S₁ and mung bean nuclease, which have a relatively broad specificity against single-stranded DNA and RNA.

Where the hybrid-binding reagent is an antibody, this may be obtained in any available manner such as conventional antiserum and monoclonal techniques. Antiserum can be obtained by well-established techniques involving immunisation of an animal, such as a mouse, rabbit, guinea pig or goat, with an appropriate immunogen. The immunoglobulins can also be obtained by somatic cell hybridisation techniques, also involving the use of an appropriate immunogen. The antibody reagent may also be a recombinant antibody, a chimeric antibody, or a single chain antibody. The antibody may be specific for RNA-DNA hybrids, DNA-DNA hybrids or RNA-RNA hybrids. An example of the production of anti-DNA-RNA monoclonal antibodies is given by Fliss et al. (Applied and Environmental Microbiology (1993) 59: 2698-2705). Antibodies specific for double-stranded nucleic acid may also be obtained from commercial sources. In preferred embodiments the antibody is labelled with either a detectable moiety or an immobilisable moiety. The covalent attachment of the label may be achieved by a number of well-known methods using a wide range of heterobifunctional reagents. For example, the method of Carlsson et aL (Biochem J (1978) 173: 723-737) may be used: the label is reacted with 3-[(2)-pyridyidithio]propionic acid N-hydroxysuccinimide ester (SPDP) to give a 2-pyridyl disulphide-activated label. This is mixed with an IgG antibody, and a disulphide exchange reaction yields a labelled antibody conjugate. Other approaches for labelling the antibody will be apparent to one skilled in the art. Preferred labels include the enzymes alkaline phosphatase, peroxidase, -galactosidase, nuclease P₁, nuclease S₁ and mung bean nuclease; the haptens digoxin and digoxigenin, and biotin or biotin analogues.

A wide range of suitable support materials is commercially available. These are preferably activated to allow the probe or the hybrid-capture agent to be attached. These include N-hydroxy-succinimide activated matrices from Pharmacia Biotech, such as Superose, Sepharose and Hi-Trap materials. N-hydroxy-succinimide -and hydrazide-activated Affigels are available from BioRad. A wide range of activated supports may also be obtained from Sigma, including CN-Br activated, epoxy-activated, nitrophenyl-chloroformate activated, N-hydroxy-succinimidyl chloroformate activated, oxirane activated and polyacryl-hydrazido activated materials; thiolated materials are also available. These latter may be easily attached to the SPDP-derivatised oligonucleotides and other agents described in the foregoing by a disulphide exchange reaction. The N-hydroxysuccimide moiety reacts with amino groups introduced into an oligonucleotide during synthesis, or with amino groups on the surface of antibodies or nucleic acid binding proteins. In addition, agarose linked to avidin, streptavidin or neutravidin is available ready-prepared from Pierce. A magnetic support material may also be usefully employed. A preferred embodiment includes a retrievable support comprising magnetic beads characterised in their ability to be substantially homogeneously dispersed in a sample medium. Preferably, the magnetic beads contain primary amine functional groups that facilitate covalent binding or association of a probe entity to the magnetic support particles. Preferably, the magnetic support beads are single domain magnets and are super paramagnetic exhibiting no residual magnetism.

A magnetic bead suitable for application to the present invention includes a magnetic bead containing primary amine functional groups marketed under the trade name BIO-MAG by Advanced Magnetics, Inc. Beads having reactive amine functional groups can be reacted with polynucleotides to covalently affix the polynucleotide to the bead. The beads are reacted with 10 percent glutaraldehyde in sodium phosphate buffer and subsequently reacted in a phosphate buffer with ethylene-diamine adduct of the phosphorylated polynucleotide.

Alternatively, the support material may itself serve as the hybrid-capture agent. The support may be an anionic exchange resin able to bind nucleic acids through a charge interaction. Suitable resins include DEAE-Sephadex, DEAE-Cellulose, a Mono-Q resin and the like. The support may also be of glass wool, glass beads or other silicaceous material well known for binding nucleic acids. Other materials able to bind nucleic acids may also be used. Commercially available materials include Biospin columns from BioRad; Nucleon QC, EasyPrep and Microspin columns from Amersham-Pharmacia Biotech; Wizard systems from Promega; and Xtreme kits from Pierce.

Particularly attractive applications, which illustrate the operation of the present invention, are described below.

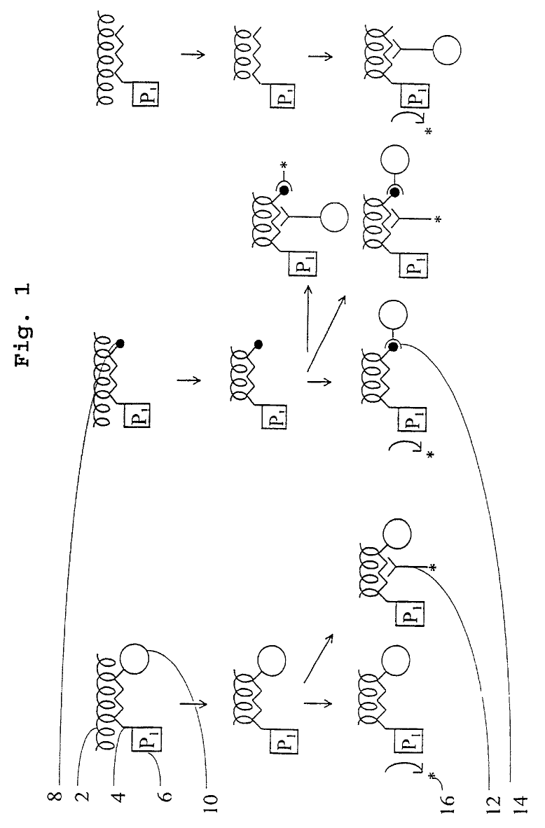

Referring now to FIG. 1, which shows three particularly preferred embodiments of the present invention, the first row shows the target nucleic acid (2), denatured if necessary to render it single-stranded, being contacted under hybridisation conditions with a nucleic acid probe (4) having a sequence complementary to at least part of the target nucleic acid and labelled at its 5′-end with an enzyme reagent (6), preferably nuclease P₁. In the first embodiment, shown in the first column, nucleic acid probe (4) is attached to support material (10). In the second embodiment, shown in the 2nd column, nucleic acid probe (4) is additionally labelled at its 3′-end with a first member of a specific binding pair (8), preferably biotin.

In the second row of FIG. 1, the pH of the mixture is adjusted to allow enzyme reagent (6) to remove single-stranded nucleic acids. These single-stranded nucleic acids comprise unhybridised probe and unhybridised target.

In the final row of FIG. 1, various options for the capture and detection of the hybrid are shown.

In the first embodiment (shown in the first column of FIG. 1), the hybrid, attached to support material (10), is introduced into a suitable column and washed with a washing agent, preferably TBS-Tween. This elutes hydrolysed materials. The hybrid is detected directly through enzyme reagent (6) or indirectly through a binding agent specific for the hybrid (12).

In the second embodiment (shown in the second column of FIG. 1), the hybrid is captured onto a support material (10), either through a second member of a specific binding pair (14), or through a hybrid-binding agent (12). The hybrid-binding agent may be relatively specific for the hybrid, preferably antibody specific for double-stranded DNA, or relatively non-specific, preferably silica. The hybrid is detected directly through enzyme reagent (6), indirectly through a second member of a specific binding pair (14), or indirectly through a hybrid-binding agent (12) specific for the hybrid and labelled with a detectable moiety.

In the third embodiment, shown in column 3 of FIG. 1, the hybrid is captured onto a support material (10) through a hybrid-binding agent (12), and detected directly through enzyme reagent (6). The hybrid-binding agent may be relatively specific for the hybrid, preferably antibody specific for double-stranded DNA, or relatively non-specific, preferably silica.

In FIG. 1, the nuclease P₁ is shown to be joined directly to the nucleic acid probe. The link may also be a n indirect one: for example: embodiments are envisaged in which the probe is labelled with a moiety, such as flourescein isothiocyanate, and nuclease P₁ is attached thereto by means of an anti-FITC antibody labelled with nuclease P₁.

Other embodiments of the invention employing the principles described above will be obvious to one skilled in the arts.

A kit for carrying out the described methods according to the present invention contains a sbm specific for the hybrid or a moiety present on the nucleic acid probe attached to a support material, optionally contained in a column, a nucleic acid probe that is complementary to the target nucleic acid to be detected and which is labelled with an enzyme reagent specific for single-stranded nucleic acids, and a detection system.

The following examples illustrate various further aspects of the operation of the invention. These examples are not intended to limit the invention in any way.

### EXAMPLE 1

Standardisation of Nuclease P₁.

Nuclease P₁ (1 mg; obtained from Sigma Chemical Company, batch no: 107F0799) was dissolved in 1 ml of water to give a concentration of 22.7 M and stored at 4° C. The activity of this solution was assayed in the following mixture: 0.16 mM NADH, 1 mM ATP, 1 mM PEP, 1 mM MgSO₄, 20 mM KCl, 0.5 mM adenosine 3′, 5′-bisphosphate, 1 U pyruvate kinase, 1 U lactate dehydrogenase and 1 U myokinase in 50 mM HEPES buffer, pH 7.2, in a total volume of 1 ml. From the change in absorbance at 340 nm the activity of nuclease P₁ was solution was found to be 320 U/ml, assuming a molar extinction coefficient of 6220 for NADH.

### EXAMPLE 2

Amplification Assay of Nuclease P₁ and Nuclease S1

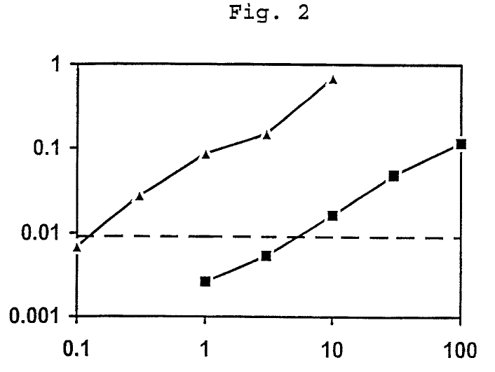

A solution of nuclease P₁ standardised according to Example 1 was serially diluted in 50 mM citrate buffer adjusted to pH 6.5 with NaOH. The assay mixture contained 20 mM 3′FADP, 0.1 mM 4-aminoantipyrine, 2 mM DHSA, 1 g horseradish peroxidase, 0.1 M glucose and 0.1 M apoglucose oxidase in a total volume of 0.1 ml. The change in absorbance was monitored at 520 nm in a Dynatech MR7000 plate reader fitted with a thermostatically controlled plate holder set to 25° C. FIG. 2 shows the performance of the nuclease P₁ assay. After a 15 minute assay period, the detection limit (defined as 3 times the standard deviation of the background reading) was 0.2 amol. Nuclease S1 was assayed in a similar manner, and the detection limit was 4 amol (FIG. 2).

### EXAMPLE 3

Oligonucleotide Synthesis

Oligonucleotides were synthesised on a Cyclone™ DNA synthesiser using the Expedite™ chemistry.

The DNA to be labelled with nuclease P₁ was complementary to a region in the middle of the ribonuclease gene containing the K66E mutation. This probe was derivatised at the 5′ end with a trityl-hexyl thiol group to facilitate linkage to nuclease P₁. The sequence was:

5′-GGTCACCTGCGAAAACGGGCAGG-3′

Another oligonucleotide specific for repeat regions of the genomic DNA of Streptococcus pneumoniae (SEQ ID No 6 of U.S. Pat. No. 5,656,432) and having the sequence:

5′-TATYYACARYSTCAAAAYAGTG-3′

and having a biotinylated 5′-end and an FITC-labelled 3′-end was obtained from Cruachem Ltd. The same oligonucleotide sequence having a thiolated 5′-end and a biotinylated 3′-end was also obtained.

The oligonucleotides were freeze-dried and stored at 4° C. until required.

### EXAMPLE 4

Derivatisation of Nuclease P₁

Nuclease P₁ (5 mg) was dissolved in 0.5 ml 0.1 M sodium bicarbonate pH 7.5 containing 0.1 M sodium chloride and desalted by gel filtration on Sephadex G25 (NAP-5 column, Pharmacia) equilibrated with the same buffer. This enzyme solution was incubated with a 50-fold molar excess of 3-(2)-pyridyldithio)-propionic acid N-hydroxysuccinimide ester (SPDP) at room temperature for 30 minutes. Unreacted SPDP was removed by gel filtration on Sephadex G25 (NAP 10 column, Pharmacia) equilibrated with the bicarbonate buffer. The 2-pyridyl disulphide-activated nuclease P₁ was stored at 4° C.

### EXAMPLE 5

Conjugation of Nuclease P₁ to an Oligonucleotide

Nuclease P₁ was linked to 2-pyridyl disulphide as described in Example 4 and stored in 0.1 M sodium bicarbonate, pH 7.5, containing 0.1 M sodium chloride at 4° C. The K66E or the thiolated S pneumoniae oligonucleotide of Example 3 was dissolved in 0.5 ml 0.1 M sodium bicarbonate buffer, pH 7.5, containing 0.1 M sodium chloride to give a final concentration of 0.36 M. This was incubated with activated nuclease P₁ prepared according to Example 4 at a mole ratio of 1:2 at room temperature for 45 minutes, followed by an incubation at 4° C. for 16 h.

The conjugate was transferred to 20 mM bis-Tris propane buffer, pH 7.5, containing 1 mM CHAPS by chromatography on Sephadex G25, and purified by ion-exchange chromatography on a Pharmacia Mono Q column. A sodium chloride gradient in the same buffer was used applied to the column and the conjugate was eluted at a molar concentration of 0.25 M.

### EXAMPLE 6

Detection of S pneumoniae genomic DNA.

Genomic DNA from S pneumoniae was extracted and treated with Pstl to break the DNA up into fragments. 95 l of the treated DNA is mixed with 10 l 1 M sodium hydroxide and incubated at room temperature for 10 minutes to denature the DNA before neutralisation with 8 l of 0.5 M sodium citrate buffer, pH 3.0, containing 2.21 M sodium chloride and 0.1% Tween 20. 50 l (34 fmol) of the S pneumoniae probe labelled with Nuclease P₁ described in Example 5, dissolved in 0.1 M Tris-HCI buffer, pH 7.5, containing 7 mM zinc sulphate, 1% (w/v) PVP, 0.1% N-lauroylsarkosine and 150 mM sodium chloride, is added. After hybridisation at 40° C. for 1 hour, the pH is adjusted to about 5.0 by the addition of acetate buffer, and the temperature maintained at 40° C. for 10 minutes, after which time more than 95% of unhybridised reporter probe will be hydrolysed.

The mixture is then introduced into to a commercial immobilised streptavidin column (Pierce ImmunoPure Immobilised Streptavidin) of volume 0.20 mL incubated with TBS. After incubation at 40° C. for 30 minutes, the column was eluted with 10 mL of 20 mM Tris-HCL buffer, pH 7.5, containing 7 mM zinc sulphate, 1% (w/v) PVP, 0.1% N-lauroylsarkosine and 150 mM sodium chloride.

The amount of hybrid captured on the column is quantified using the amplification assay described in Example 2: 0.1 mL of the reaction mixture is introduced onto the column, and allowed to produce a coloured product. This is eluted by introducing more of the 20 mM Tris-HCI buffer, pH 7.5, containing 7 mM zinc sulphate, 1% (w/v) PVP, 0.1% N-lauroylsarkosine and 150 mM sodium chloride, and its absorbacnce measured.

This column-based washing and detection step may be conveniently automated using the DuoPrep from Pharmaceutical Technology. This allows the washing and detection solutions to be pumped onto and off the column in a programmed fashion, allowing the coloured product to be recovered in a small volume.

Industrial Applicability

Accordingly, it will be seen that the method of the present invention can be used to detect hybrids formed between a target nucleic acid and a nucleic acid probe labelled with an enzyme reagent which removes single-stranded nucleic acid. This approach eliminates the possibility of cross-talk arising out of the binding of sbm to any single-stranded nucleic acid present. This means that the complex formed between hybrid and sbm can be detected using highly sensitive approaches, such as enzyme amplification or chemiluminescence. In addition, the nucleic acid probe may be labelled with nuclease P₁ at each end, thereby giving an increase in the overall sensitivity of the detection reaction.

The method has the additional advantage that it utilises a single probe, which offers cost savings and simplifies the design of assay protocols.

Although the description above contains many specificities, these should not be construed as limiting the scope of the invention but as merely providing illustrations of some of the presently preferred embodiments of this invention. For example, the nucleic acid probe may be a peptide nucleic acid probe, or another nucleic acid analogue having modified bases or an altered backbone. When the nucleic acid probe is a peptide nucleic acid probe the enzyme reagent may be a protease specific for single stranded peptide nucleic acid.

Thus the scope of the invention should be determined by the appended claims and their legal equivalents, rather than by the examples given in the specification.

1. A method for detecting a single-stranded target nucleic acid comprising the steps of:
(a) forming a hybrid between a target nucleic acid and a nucleic acid probe, said nucleic acid probe labelled with an enzyme reagent which hydrolyses single-stranded nucleic acid but is substantially without effect on double-stranded nucleic acid, said hybrid formed under conditions of pH which are outside the activity range of said enzyme reagent,
(b) adjusting said pH to a value within the activity range of said enzyme reagent, whereby said enzyme reagent substantially hydrolyses any single-stranded nucleic acid present; and
(c) contacting said hybrid with a detection reagent to detect the hybrid, characterised by, prior to step (c), bringing the nucleic acid probe or hybrid into contact with a solid support to attach it thereto or bringing the nucleic acid probe or hybrid into contact with a capture reagent, optionally linked to a solid support, to capture the nucleic acid probe or hybrid; and washing the capture reagent or solid support on which the hybrid is immobilised with a washing fluid while the capture reagent or solid support is contained within a vessel that is adapted to retain the capture reagent or solid support but not to retain fluid in which the capture reagent or solid support is dispersed, whereby material which has not been captured by the capture reagent or otherwise immobilised on a solid support is eluted from the vessel.

2. A method as claimed in claim 1, wherein the hybrid is contacted with a capture reagent, optionally linked to a solid support, contained in the vessel.

3. A method as claimed in claim 1, wherein in step (a) the nucleic acid probe is attached to a solid support which is subsequently introduced into the vessel.

4. A method as claimed in claim 1, wherein the nucleic acid probe in step (a) is attached to a solid support contained within said vessel prior to performing step (b) of the method.

5. A method according to any of claims 1-4 wherein said enzyme reagent is detectable, whereby said hybrid is detected.

6. A method according to any of claims 1-5 wherein said enzyme reagent is a nuclease.

7. A method according to claim 6 wherein said nuclease is selected from the group consisting of: ribonuclease A and ribonuclease T1 in combination, exodeoxyribonuclease I (E.C. 3.1.11.1), mammalian DNase III, exonuclease IV, T2-and T4-induced exodeoxyribonucleases, exodeoxyribonuclease (phage sp3-induced) (E.C. 3.1.11.4), exodeoxyribonuclease V (E.C. 3.1.11.5), Haemophilus influenzae ATP-dependent DNase, exodeoxyribonuclease VII (E.C. 3.1.11.6), Micrococcus luteus exonuclease, exoribonuclease II (E.C. 3.1.13.1), RNase Q, RNase BN, RNase PIII, RNase Y, venom exonuclease (E.C. 3.1.15.1), hog kidney phosphodiesterase, Lactobacillus exonuclease, spleen exonuclease (E.C. 3.1.16.1), Lactobacillus acidophilus nuclease, B subtilis nuclease, deoxyribonuclease IV (phage T4-induced) (E.C. 3.1.21.2), DNase V (mammalian), Aspergillus sojae DNase, B subtilis endonuclease, T4 endonuclease III, T7 endonuclease I, Aspergillus DNase K2, Vaccinia virus DNase VI, yeast DNase, Chlorella DNase, Aspergillus deoxyribonuclease K1 (E.C. 3.1.22.2, Aspergillus nuclease S1 (E.C. 3.1.30.1), N crassa nuclease, mung bean nuclease, and Penicillium citrinum nuclease P1.

8. A method according to any of claims 1-4 wherein said enzyme reagent is nuclease P1 or nuclease S1.

9. A method according to any of claims 1 or 2 wherein said capture reagent is a hybrid-binding reagent.

10. A method according to claim 9 wherein said hybrid-binding reagent is an antibody specific for double-stranded nucleic acid or a DNA-binding protein specific for double-stranded nucleic acid.

11. A method according to claim 10 wherein said antibody is selected from the group consisting of monoclonal antibody, polyclonal antibody, recombinant antibody, chimeric antibody and single-chain antibody.

12. A method according to claim 9 wherein said hybrid-binding reagent is labelled.

13. A method according to any of claims 1 to 12 wherein said nucleic acid probe additionally comprises a first member of a specific binding pair.

14. A method according to claim 13 wherein said first member is selected from the group consisting of digoxin, digoxygenin, fluorescein, fluorescein isothiocyanate and biotin.

15. A method according to claims 1 or 2 wherein said capture reagent comprises a second member of a specific binding pair.

16. A method according to claims 1 to 4 wherein said detection reagent comprises a second member of a specific binding pair.

17. A method according to claims 15 or 16 wherein said second member is selected from the group consisting of anti-digoxin antibody, anti-digoxygenin antibody, anti-fluorescein antibody, anti-fluorescein isothiocyanate antibody, avidin, streptavidin and neutravidin.

18. A method according to claim 17 wherein said second member has a label.

19. A method according to claim 18 wherein said label is a detectable label.

20. A method according to claim 19 wherein said detectable label is selected from the group consisting of enzyme, fluorescent moiety, chemiluminescent moiety, and electrochemiluminescent moiety.

21. A method according to claim 20 wherein said enzyme is -galactosidase or horseradish peroxidase.

22. A method according to claim 20 wherein said enzyme is selected from the group consisting of alkaline phosphatase, nuclease P1 and nuclease S1.

23. A method according to any of the preceeding claims wherein said detection reagent comprises an amplification system.

24. A method according to claim 23 wherein said amplification system comprises an apoenzyme which is convertible into a holoenzyme by interaction with an accessory subunit; and a masked form of said subunit which is convertible into its active unmasked form by the action of the said enzyme.

25. A method according to claim 24 wherein said subunit is FAD and said masked form is 3′FADP.

26. A method according to claim 24 or 25 wherein said apoenzyme is apo-glucose oxidase or apo-D-aminoacid oxidase.

27. A method according to any of the preceding claims wherein said target nucleic acid is isolated from a test sample.

28. A method according to any of the preceding claims wherein said target nucleic acid is produced by a target amplification means.

29. A method according to claim 30 wherein said target amplification means is selected from the group comprising polymerase chain reaction, ligase chain reaction, nucleic acid sequence-based amplification, cycling probe amplification and strand displacement amplification.

30. A method according to any of the preceding claims wherein said target nucleic acid is selected from the group consisting of DNA, RNA or PNA.

31. A method according to any of the preceding claims wherein said probe nucleic acid is selected from the group consisting of DNA, RNA or PNA.

32. An assay kit for detecting a single-stranded target nucleic acid comprising a nucleic acid probe complementary to the target nucleic acid to be detected which is labelled with an enzyme able to substantially hydrolyse single-stranded nucleic acid but not double-stranded nucleic acid, a capture reagent optionally linked to a solid support, and a detection reagent.

33. The assay kit according to claim 32 wherein said nuclease is selected from the group consisting of: ribonuclease A and ribonuclease T1 in combination, exodeoxyribonuclease I (E.C. 3.1.11.1), mammalian DNase III, exonuclease IV, T2-and T4-induced exodeoxyribonucleases, exodeoxyribonuclease (phage sp3-induced) (E.C. 3.1.11.4), exodeoxyribonuclease V (E.C. 3.1.11.5), Haemophilus influenzae ATP-dependent DNase, exodeoxyribonuclease VII (E.C. 3.1.11.6), Micrococcus luteus exonuclease, exoribonuclease II (E.C. 3.1.13.1), RNase Q, RNase BN, RNase PIII, RNase Y, venom exonuclease (E.C. 3.1.15.1), hog kidney phosphodiesterase, Lactobacillus exonuclease, spleen exonuclease (E.C. 3.1.16.1), Lactobacillus acidophilus nuclease, B subtilis nuclease, deoxyribonuclease IV (phage T4-induced) (E.C. 3.1.21.2), DNase V (mammalian), Aspergillus sojae DNase, B subtilis endonuclease, T4 endonuclease III, T7 endonuclease I, Aspergillus DNase K2, Vaccinia virus DNase VI, yeast DNase, Chlorella DNase, Aspergillus deoxyribonuclease K1 (E.C. 3.1.22.2, Aspergillus nuclease S1 (E.C. 3.1.30.1), N crassa nuclease, mung bean nuclease, and Penicillium citrinum nuclease P1.

34. An assay kit according to claim 32 wherein said enzyme reagent is nuclease P1 or nuclease S1.

35. An assay kit according to any of claims 32 to 34 wherein said capture reagent is a specific binding member specific either for hybrids formed between said single-stranded target nucleic acid and said nucleic acid probe or for a moiety present on said nucleic acid probe.

36. An assay kit according to claim 35 wherein said specific binding member is an antibody specific for double-stranded nucleic acid or a DNA-binding protein specific for double-stranded nucleic acid.

37. An assay kit according to claim 36 wherein said antibody is selected from the group consisting of monoclonal antibody, polyclonal antibody, recombinant antibody, chimeric antibody and single-chain antibody.

38. An assay kit according to claim 35 wherein said moiety is selected from the group consisting of digoxin, digoxygenin, fluorescein, fluorescein isothiocyanate and biotin.

39. An assay kit according to claim 35 wherein said specific binding member is selected from the group consisting of anti-digoxin antibody, anti-digoxygenin antibody, anti-fluorescein antibody, anti-fluorescein isothiocyanate antibody, avidin, streptavidin and neutravidin.

40. An assay kit according to any of claims 35 to 39 additionally comprising a detection system.

41. An assay kit according to claim 40 wherein said detection system is an amplification system.

42. An assay kit according to claim 41 wherein said amplification system comprises an apoenzyme which is convertible into a holoenzyme by interaction with an accessory subunit; and a masked form of said subunit which is convertible into its active unmasked form by the action of the said enzyme.

43. An assay kit according to claim 42 wherein said subunit is FAD and said masked form is 3′FADP.

44. An assay kit according to claim 42 or 43 wherein said apoenzyme is apo-glucose oxidase or apo-D-aminoacid oxidase.

45. A method according to claims 1 to 4 wherein said solid support is selected from the group consisting of: agarose, derivatised agarose, actlamide, and derivatised acrylamide.

46. A method according to claims 1 to 4 wherein said solid support comprises a magnetic bead.

47. A method according to claims 1 to 4 wherein said solid support comprises an anionic exchange resin.

48. A method according to claims 1 to 4 wherein said solid support is selected from the group consisting of: glass, glass wool and silica.

49. A method as claimed in any preceding method claim, wherein the vessel is a column.
